(19) United States
(12) Patent Application Publication
(10) Pub. No.: US 2005/0005717 A1
(43) Pub. Date: Jan. 13, 2005

(54) Probe holder

(75) Inventors: Marc Pensis, Preston, CT (US); Wayne Byassee, North Stonington, CT (US); Laurie A. St. Pierre Berry, Carolina, RI (US)

Correspondence Address: PFIZER INC, 150 EAST 42ND STREET, 5TH FLOOR - STOP 49, NEW YORK, NY, 10017-5612, US

(73) Assignee: Pfizer Inc, New York, NY (US)

(21) Appl. No.: 10/847,060

(22) Filed: May 17, 2004

Related U.S. Application Data
(63) Continuation-in-part of application No. 10/616,902, filed on Jul. 10, 2003.

(51) Int. Cl. G01N 21/85; G01N 33/15

(52) U.S. Cl. 73/866.5; 422/108; 250/338.1; 73/866

(57) ABSTRACT

A near infrared (NIR) probe is disposed in a holder which is mounted within the wall of an enclosed process vessel. The probe reads and determines the moisture content of a product undergoing processing or drying from the enclosed vessel. The probe holder is formed with conduits, and a gas, such as air or nitrogen, is transmitted in periodic high pressure pulses through the holder conduits and transversely across the probe detecting end face to clear the probe end face of moisture and particulate contaminants to provide accurate real time moisture content determinations. The NIR probe and holder are disposed in a fluid tight construction.

### PRIOR RELATED APPLICATIONS

This application is a continuation-in-part application of Ser. No. 10/616,902, filed Jul. 10, 2003.

### BACKGROUND OF THE INVENTION

1. Field of the Invention

This invention relates to holders for detecting probes. This invention also relates to a system and method for the accurate detection of a substance with a probe. In a more specific respect, this invention relates to a holder, system and method for making accurate process material moisture content determinations with infrared and near infrared probes. This invention also relates to a system and method that provides high accuracy moisture content determination in a process mass, and to consequently automatically control process operation and parameters.

2. Background and Discussion of the Prior Art

Infrared probes are used to detect moisture content in various production operations, such as in paper mill processing and wood drying. Infrared probe readings are often inaccurate in that moisture or contaminants collect on the detecting end of the probe thereby introducing errors in the moisture content reading.

In pharmaceutical process operations, it is important to accurately determine the moisture content of the process material in dryers and processing vessels for controlled drying or processing. Over drying adversely impacts on crystal size in pharmaceutical crystals. Crystal size and shape are important in pharmaceutical products. Accurate moisture content determination in pharmaceutical crystals is therefore imperative. The present pharmaceutical process technique for product moisture content determination entails use of sample collectors. In this process technique, the operator periodically opens the otherwise enclosed process vessel and uses the sample collector to collect a sample from the product mass or slurry. The collector with sample is taken to a laboratory for moisture content analysis. The moisture content determination is then communicated to the process operator for process control adjustment, or even shutdown. This technique is unduly time consuming, labor intensive and necessitates disruptive entry into the closed process vessel. Further, this moisture content determination is not in real processing time. Further, the disruptive sample removal from the otherwise closed process vessel may introduce outside moisture or contaminants with consequential additional error in the moisture product content determination or adverse product characteristics.

U.S. Pat. No. 6,395,538, issued May 28, 2002 to Naughton et al. discloses a method and system for providing real-time, in situ bio-manufacturing and control in response to IR spectroscopy. Naughton et al. discloses spectroscopic monitoring of a biomolecule to determine the different stages in a bio-manufacturing process.

The pharmaceutical manufacturing field desires a direct, online, real time, rapid and accurate means for moisture content determination of pharmaceutical product in dryers and other process vessels. This is particularly desired for determining moisture content of such pharmaceutical products.

The pharmaceutical manufacturing art desires product moisture content readings, which are effectively free of contaminant and ambient moisture induced error, process disruption and process control interference. The present invention provides the solution to these pharmaceutical manufacturing art needs.

### SUMMARY OF THE INVENTION

A moisture detecting probe is mounted in a holder, which holder includes a gas distribution conduit to provide gas at a predetermined sufficiently high pressure across the detecting end of the probe end to clear the probe of contaminants and moisture, to thereby provide accurate moisture content determinations.

The detecting probe and holder combination of the present invention, in one preferred embodiment, is disposed in the wall of an enclosed process vessel, dryer or chamber to determine the moisture content of a product undergoing processing or drying. A near infrared probe is preferably used to detect the moisture content. A controller is operably connected to the vessel so that at predetermined periodic intervals, just prior to each desired moisture content determination, a high-pressure pulse of gas, such as air or nitrogen, is provided to the holder conduits and transversely across the detecting end face of the near infrared probe. The high-pressure pulsed gas assuredly clears the probe detecting end of residual moisture and/or particulates. The controller then immediately actuates the probe for a real time accurate moisture content reading.

The gas may be initially provided by a supply controller at a constant low pressure purge of no more than about 10 psi, and immediately prior to the probe reading, the controller is actuated to supply the gas at a higher pressure of about 10 to 45 psi or more to assuredly clear the probe detecting end. The high-pressure gas purge is then terminated. A master controller actuates the probe immediately after the high-pressure gas purge is terminated. The low-pressure gas purge is then reinstituted until just prior to the next desired predetermined probe reading.

In a more specific preferred embodiment, the present invention includes one or more of the foregoing inventive features in operable combination with a pharmaceutical process vessel or dryer for an accurate real time determination of the moisture content of the pharmaceutical process material or product undergoing processing or drying. In one particular embodiment, the holder and probe are mounted in the process vessel adjacent the pharmaceutical product mixer blades so that the probe optimally faces the pharmaceutical product. The master controller provides a signal, based on the probe moisture content determination, to diverse process controllers (e.g. temperature, pressure, mixer rpm) to accordingly vary the process parameters to ensure that the pharmaceutical product has the desired product specifications. This real time accurate probe readings affect a closely monitored and controlled process with concomitant controlled pharmaceutical product specifications.

### BRIEF DESCRIPTION OF THE DRAWINGS

FIG. 1 is a perspective exploded view of the probe holder and probe;

FIG. 2 is a sectional view of the probe holder taken along line 2-2 of FIG. 1, with the probe disposed in the holder;

FIG. 3 is a proximate end view of the coupler portion of the probe holder;

FIG. 4 is a proximate end perspective partial sectional view of the probe holder;

FIG. 5 is a schematic diagram of the system and method of the present invention; and

FIG. 6 is a schematic diagram of a second embodiment of the system and method as shown in FIG. 5.

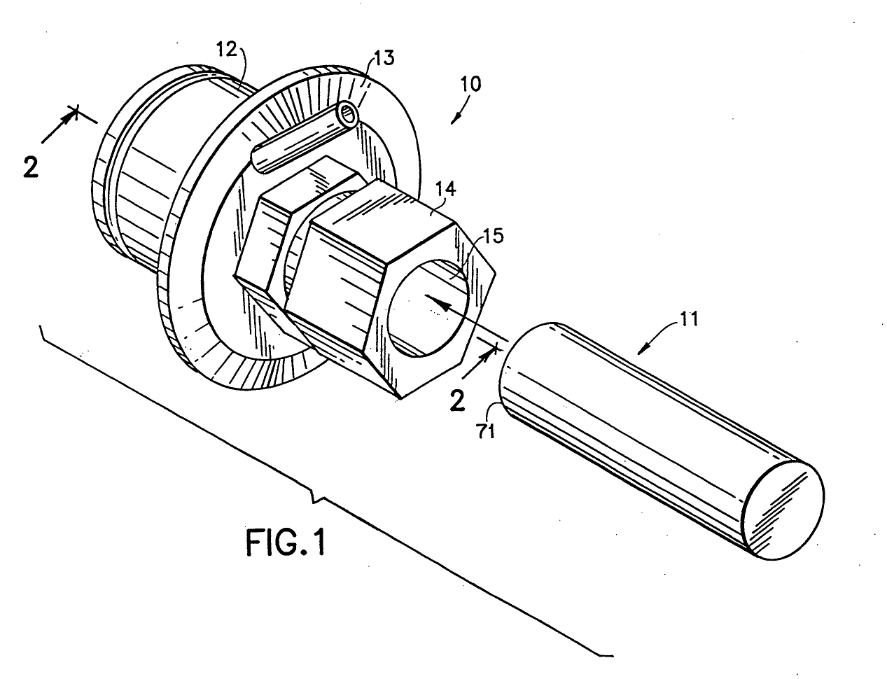

### DESCRIPTION OF THE PREFERRED EMBODIMENT

The terms “near infrared” and “NIR” as used hereinbefore and hereinafter throughout the specification and claims refer to wavelengths of between about 1100 and 2200 nm.

Referring to the FIGS., there is shown probe holder 10 and near infrared probe 11. Probe holder 10 is, in general terms, an integral assembly constructed of distally disposed tubular member or coupler 12, annular flange or manifold 13, and proximately disposed tubular lock member 14. Holder 10 slidably receives probe 11 in central orifice 15. Holder 10 is generally constructed of machined metal components, namely, coupler 12, flange 13 and lock member 14 which are slidably inter-fitted and welded in an integral construction.

Coupler 12 has a distally disposed annular end face 16, cylindrical outer wall 17 with distally disposed annular groove 18, a proximately disposed annular end wall 19, a first inner cylindrical wall 20 terminating in end wall 21, a second inner cylindrical wall 22 which forms a portion of central orifice 15, and a distally disposed cylindrical wall 92. End wall or lip 23 is disposed between and contiguous with walls 22 and 92. End wall 23 functions as an abutment or seat for the distal end 71 of probe 11. Probe 11, when seated against end wall 23, is in fluid tight disposition in holder 10 by means of a deformable swage lock (not shown) of well known construction, disposed between probe 11 and holder central orifice 15. An O-ring (not shown) can additionally be provided to insure the fluid tight seal of the probe in the holder. Other fluid tight mechanical sealing means known in the art may likewise be used to securely hold probe 11 in holder central orifice 15.

A series of three spaced holes 25 (typical) are drilled or formed in holder 10. Holes 25 are circumferentially disposed at 120°, and equally radially disposed with respect to central orifice axis 26. Holes 25 extend from coupler proximately disposed wall 19 to coupler distally disposed end face 16. A distal end weld plug 27 (typical) forms a blind hole for each respective hole 25. A series of three cross-holes 28 (typical) are drilled in holder 10. Cross-holes 28 are circumferentially disposed and extend from outer cylindrical surface 17 to inner cylindrical wall 92. Each cross-hole 28 intersects and communicates with a respective hole 25. Cross-hole 28 terminate in a respective end opening 30 in wall 92. Each cross-hole 28 has a radially disposed central axis 31 which is perpendicularly disposed to and intersects central orifice axis 26. A plug weld 32 (typical) forms a blind hole for each cross-hole 28. In this manner of construction, holes 25 are contiguous with respective cross-holes 28 to form respective channels or conduits for the simultaneous distribution of a gas in a radially inward direction through end openings 30, for purposes hereinafter appearing.

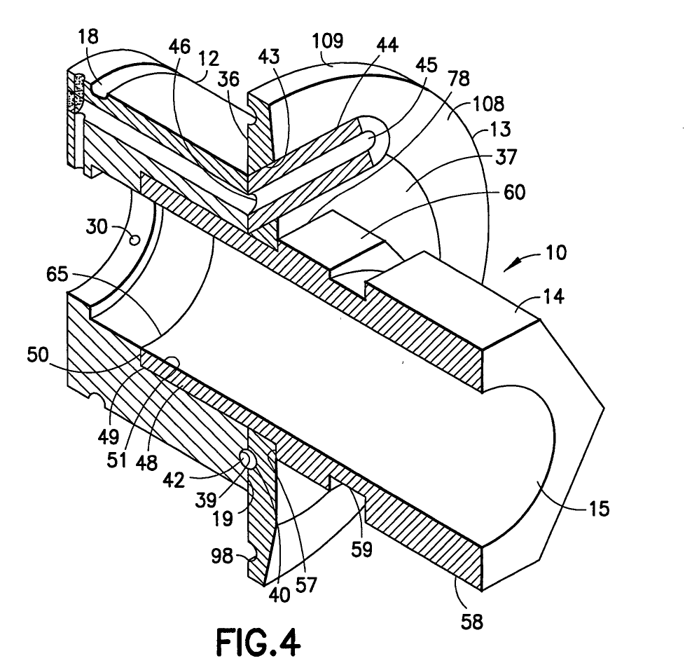

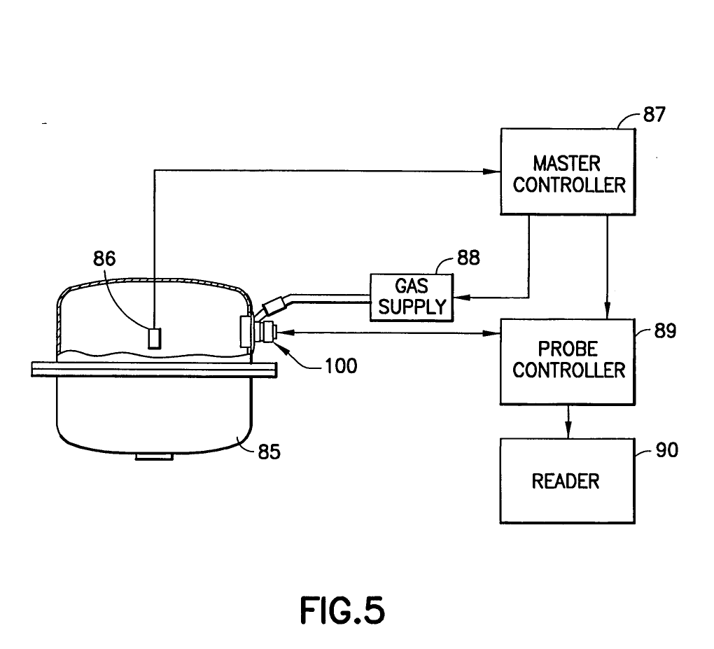

Coupler proximately disposed annular end wall 19 is formed with an annular semi-circular groove 39 which is contiguous with the distal end opening 35 (typical) of each respective hole 25. Flange 13 is formed with a distal wall 36, proximately radially inwardly disposed wall 37 and contiguous outwardly disposed wall 108. Contiguous walls 37 and 108 provide a frustoconical configuration which is conjoined to distal wall 36 by peripheral cylindrical wall 109 (FIG. 4). An annular semi-circular groove 40 is formed in flange distal wall 36 which is similarly sized to groove 39 so that when wall 36 abuts wall 19, complimentary grooves 39 and 40 form annular channel or orifice 42. An angularly disposed hole 43 is drilled in flange 13 to receive gas supply hose 44. Hose 44 has an inlet 45 and an outlet 46. Hose outlet 46 is disposed within flange 13 and is contiguous with annular channel 42 and one of the coupler holes 25. Hose 44 is welded to flange 13 at circular mating corner 74. In this manner of construction, gas enters hose inlet 45, passes through hose 44 and hose outlet 46 into annular orifice 42, and then simultaneously distributed through each hole 25 to each cross-hole 28 and in turn to each respective end opening 30.

Lock member 14 Is formed with a distally extending cylindrical portion 48 having an outer surface or wall 49, transversely disposed distal end wall 50 and an inner cylindrical wall 51. Lock member end wall 50 abuts coupler end wall 21. Lock member inner cylindrical wall 51 is flush with coupler inner cylindrical wall 22 to form the full length of central orifice 15. A weld (not shown) is made at inner joining line 65 to provide an integral coupler and lock member construction (FIG. 4).

Flange 13 is formed with inner cylindrical wall 54 which is sized to slidably receive lock member outer wall 49. With flange 13 disposed between lock member 14 and conjoined to coupler 12, flange distal wall 36 abuts coupler wall 19, and flange proximate wall 37 abuts lock member wall 57. Lock member 14 is formed with proximate hexagonal outer peripheral portion 58, cylindrical portion 59, and hexagonal portion 60. Flange 13 is fixedly seated between lock member hexagonal portion 60 and coupler proximate end wall 19. Welds (not shown) are provided respectively at hexagonal mating line 78 and circular mating line 79 to provide an integral coupler, flange and lock member construction.

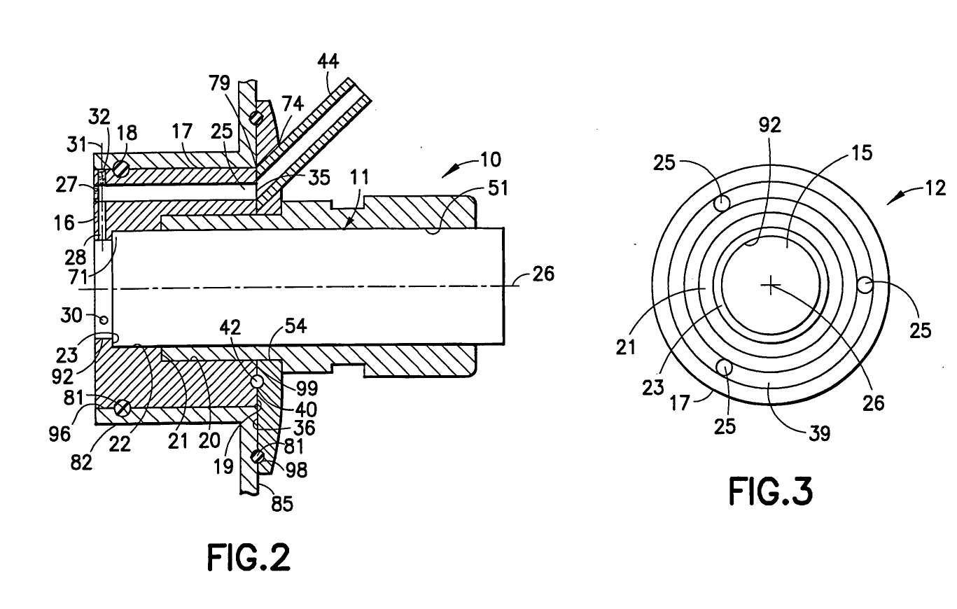

Coupler circumferential groove 18 and flange annular groove 98 are sized to receive respective O-rings 81 for fluid tight engagement within vessel wall mount or collar 82 of process vessel or chamber 85 (FIG. 2). Other fluid tight sealing means well known in the process vessel art are also within the contemplation of the invention.

With specific reference to FIG. 2, there is shown the probe holder annular end face 16 is in flush alignment with the process vessel wall flange face. The flange is briefly secured to and forms a permanent part of the process vessel wall.

Referring specifically to FIGS. 2 and 5, the enclosed vessel or chamber 85 has an integral collar 82, which is formed with sleeve 96 into which holder 10 with probe 11 are slidably received, thereby forming assembly 100. It is also within the contemplation of the present invention to first slidably insert, or otherwise seat the probe 11 in holder 10, and then insert the assembled probe and holder into the wall of the process vessel. Assembly 100 is in fluid tight construction with respect to the process vessel or chamber 85 by means herebefore described or by other vessel fluid tight constructions well known in the process vessel art. Gas supply controller 88 supplies gas to assembly 100 through hose 44 and holder 10, and in turn across the detecting end face of probe 11. A process instrument 86 is operably disposed with respect to vessel 85, so that at a predetermined process parameter or condition such as each rotation of process slurry mixer blade, a signal is transmitted to master controller 87. Master controller 87 upon receipt of the signal actuates gas supply controller 88 to supply pressurized gas at a predetermined pressure to holder 10 to clear the probe detecting end face as hereinbefore described. Master controller 87 then immediately, after clearance of the probe detecting end face, signals probe controller 89 to take a probe reading. Probe 11 transmits the reading to probe controller 89 and in turn to a reader or recorder 90.

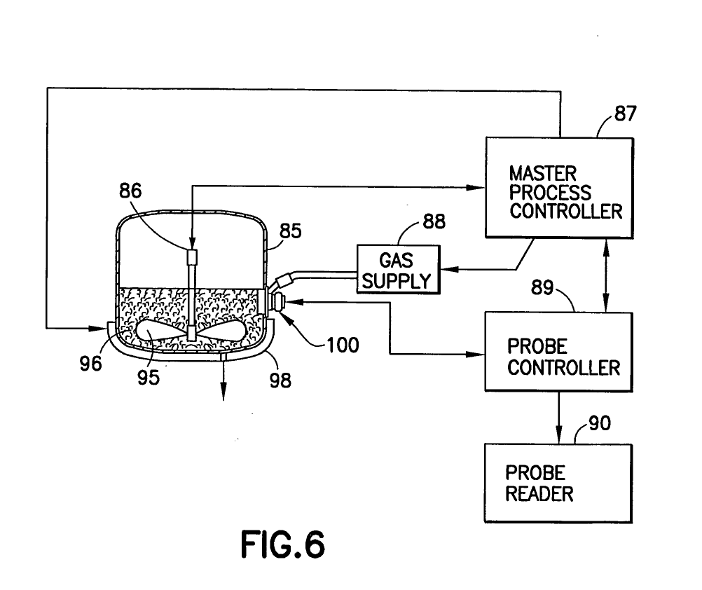

In a preferred embodiment, probe 11 is a near infrared (NIR) probe that detects the moisture content of the process material in closed process vessel 85. A mixer blade 95 rotates in the process mass or slurry (e.g. pharmaceutical crystals) undergoing processing (e.g. drying). Master controller 87 actuates a low-pressure gas (e.g. air or nitrogen) purge of about 5 to 10 psi across the end face of probe 11. Process instrument 86 detects each rotation of the slurry blade as it passes the probe 11, and accordingly at that time, immediately after the blade passes beneath the probe, signals master control 87 to actuate gas supply 88 to provide a surge or blast of high pressure gas at 10 to 45 psi or more across the sapphire crystal end face of the NIR probe to assuredly clear the probe distal end face. Master controller 87 then stops the high-pressure gas surge momentarily and simultaneously actuates a probe moisture content reading via probe controller 89. The moisture content reading may be transmitted to reader or recorder 90. The master controller 87 then actuates a low psi gas purge of about 5 to 10 psi, and repeats the probe reading cycle. In this manner, the invention provides an entirely intra-vessel product moisture content reading and determination which is free of error introduced by moisture and/or contaminants, and further provides a real time accurate moisture content determination without operator interface, undesired process disruption or process vessel opening. The process operator merely reads the reader and determines whether to adjust the process parameters (e.g., temperature) or stop the process. It is also within the contemplation of the present invention to cooperatively automatically control and adjust the process parameters, as will be further discussed in relation to the embodiment of FIG. 6.

The distal end of the probe may be disposed adjacent to the process mass or slurry, as best shown in FIG. 6. As previously discussed, the high pressure air cleans the probe of any process material or moisture disposed on the probe distal end tip to clear or clean the probe immediately prior to taking a probe reading.

While the preferred embodiment is to provide low pressure and high-pressure gas pulses, it is to be understood that other variations of gas pulses are within the contemplation of the invention. By way of example, it has been found that the low-pressure pulse may be terminated when the product crystals slurry nascent liquid is greatly diminished as when the pharmaceutical crystals drying approaches the desired end point.

The present invention is particularly useful in, but not limited to, the formation and drying of crystals in the manufacture of pharmaceuticals.

Insofar as over drying adversely impacts crystal size, close monitoring of the moisture content of the crystals slurry is important. The afore-described preferred embodiment is particularly useful in such applications. It is however within the broad contemplation of the invention to use the probe holder in diverse environments, including by way of example, process vessels, reactors and dryers. The probe holder is preferably used in a closed vessel or chamber but may also be used in processing environments, which communicate with the ambient air. Continuous as well as batch process operations are within the contemplation of the present invention.

The probe is preferably a near infrared probe of Hasteloy construction having flexible fiber optics, which provides NIR through a detecting sapphire window end face onto the product undergoing moisture content determination. One preferred commercially available probe system useful in the present invention is the XDS NIR SmartProbe Analyzer manufactured by Foss, Silver Spring, Md. 20904. While the preferred embodiment is described with respect to an NIR probe and to a pharmaceutical product moisture content determination, it is within the contemplation of the invention to use the probe holder with other probes, by way of example, infrared probes and other types of substance or condition detecting probes having a detecting end face. The invention contemplates using any gas, which is non-reactive with respect to the particular product and process. Useful gases are air, nitrogen, the inert gases (e.g. argon), and the like.

It is also within the contemplation of the present invention to provide fully automated process operations with and between the probe controllers and the master process controller, wherein, by way of example, the process parameters controllers would be automatically adjusted concomitantly with each periodic probe moisture content determination. In this further preferred embodiment, and with specific reference to FIG. 6, the master controller 87 transmits a signal to adjust a specific process operation, such as in the preferred embodiment, a heating jacket 98 on process vessel 85, and in turn consequently controls the temperature of the reaction mass 96 in process vessel 85. That is, the master process controller 87, in response to the real time accurate probe moisture content determination regulates the temperature of the heating fluid in jacket 98, which in turn regulates the temperature of the pharmaceutical product parameters (e.g., crystal size, product yield). The master controller 87 signals the heating controller to heat or discontinue heating the fluid in jacket 98 in response to the specific probe readings, thereby closely controlling the temperature of the process mass 96 in the process vessel 85 to obtain the desired product specification.

It is also within the contemplation of the present invention for the master process controller 87, in response to the probe readings, to send a signal to the mixer 86 to change the speed of the mixer blades 95 or to stop the mixing action in the process mass or slurry 96.

It is within the contemplation of the present invention for the process controller to control diverse process parameters including, without limitation, temperature, pressure, viscosity and the like. The accurate real time probe determination will provide closely controlled input signals to the process controller thereby assuring accurate closely controlled process parameters.

While the foregoing describes a preferred embodiment of the invention, it is within the ordinary skill of the practitioner to make obvious modifications and changes within the broad contemplation of the invention as set forth in the adjoined claims.

1. In combination:
a process vessel for containing a mass product comprising a substance to be detected; and
a probe, said probe having a proximate end and a distal end, said distal end comprising means for detecting a substance, and a holder having means for retaining the probe in a predetermined position with respect to the product mass, and means for providing a gas to the distal end of the probe to clear the means for detecting a substance for the accurate detection of the substance.

2. The combination of claim 1, said means for detecting a substance comprising means for the infrared detection of moisture.

3. The combination of claim 1, said means for retaining the probe comprising means for slidably receiving the probe to the predetermined.

4. The combination of claim 1, said means for retaining the probe comprising fluid tight means between the holder and the probe.

5. The combination of claim 4, said holder and probe comprise an integral construction.

6. The combination of claim 1, said holder and probe comprise an integral construction.

7. The combination of claim 1, further comprising means for mounting said probe and holder in a portion of the process vessel so that the probe is juxtaposed in the product mass comprising the substance to be detected.

8. The combination of claim 1, said combination further comprises means for controlling the process for making the product, and means for generating a signal from the probe, said signal proportionally corresponding to the detection of the amount of the substance, and wherein said probe is operably connected to the means for controlling the process so that signal actuates the means for controlling the process in response to the detection of the amount of the substance.

9. The combination of claim 1, said combination further comprising said process vessel having a wall and a mixer blade operably disposed in the process vessel for mixing the product, and wherein said holder is mounted in said vessel adjacent the mixer blade so that the probe distal end is operably facingly disposed with respect to the product mass comprising the substance to be detected.

10. The combination of claim 7, said means for mounting said probe being in flush disposition with a part of the process vessel wall.

11. In combination, an NIR probe and holder;
an NIR probe, said probe having a proximate end and a distal end, said distal end comprising means for detecting a substance, and a holder having means for retaining the probe in a predetermined position in the holder, and means for providing a gas to the distal end of the probe to clear the means for detecting a substance for the accurate detection of the substance.

12. The NIR probe and holder of claim 10, said NIR holder comprise a fluid tight integral construction.

13. A method for making accurate detection probe readings comprising:
(a) providing a probe having a detecting end;
(b) providing a holder for holding the probe so that the probe and holder are in a fluid tight disposition and held in a predetermined position in a process vessel containing a product comprising a substance for detection, said holder comprising an orifice for gas flow; and
(c) providing a gas through the orifice to the probe detecting end to clear the probe detecting end for an accurate probe reading of the substance.

14. The method of claim 13, wherein the probe detects moisture.

15. The method of claim 13, wherein step (b) further comprises providing the gas transversely to the probe detecting end.

16. The method of claim 13, further comprising repeating step (c) and wherein step (b) further comprises providing the gas intermittently between probe readings.

17. The method of claim 16, further comprising mounting said holder and probe in the wall of a process vessel.

18. The method of claim 17, said process vessel comprises a process dryer, said substance comprises moisture, and said gas comprises one selected from air, nitrogen and an inert gas.

19. The method of claim 18, wherein step (c) further comprises providing the gas at a first pressure, and then immediately prior to the probe reading, providing the gas at a second pressure higher than the first pressure.

20. The method of claim 13, further comprising generating a signal from the probe in response to the detection of the substance, transmitting the signal to means for controlling the process parameter, whereby the means for controlling the process parameter is activated, thereby controlling a process for making the product.

21. The method of claim 13, further comprising controlling a process parameter to ensure that the product is within the desired specification.

22. The method of claim 21, wherein the product is a pharmaceutical.

23. The method of claim 21, wherein the pharmaceutical comprises crystals.

24. The method of claim 23, wherein the desired specification comprises one selected from crystal size and crystal shape.

25. The method of claim 13, said probe and holder comprise a fluid tight construction.

26. The method of claim 13, said probe and holder comprise an integral construction.
